(19) United States
(12) Patent Application Publication
(10) Pub. No.: US 2009/0198093 A1
(43) Pub. Date: Aug. 6, 2009

(54) System and method for combined embolization and ablation therapy

(76) Inventors: Oliver Meissner, Munich (DE); Thomas Redel, Poxdorf (DE)

Correspondence Address: BRINKS HOFER GILSON & LIONE, P.O. BOX 10395, CHICAGO, IL, 60610, US

(21) Appl. No.: 12/012,960

(22) Filed: Feb. 6, 2008

(51) Int. Cl. A61M 36/02; A61B 18/04; A61M 25/10; A61B 18/02; A61B 17/00; A61B 8/00; A61N 7/02; H05G 1/00

(52) U.S. Cl. 600/2; 606/34; 378/197; 604/96.01; 606/28; 601/3; 606/21; 600/437; 128/898; 606/130

(57) ABSTRACT

A system and method of treating cancerous tissues using a combination of embolization and ablation therapies is described. An imaging device is used to determine the tissue volume to be treated, and the steps of embolization and ablation are performed while the patient remains on a treatment table. The embolization treatment may be combined with chemotherapy directly administered to the tumor. The position of the volume to be treated is visualized by the imaging device to monitor the progress of the treatment and to guide ablation treatment so as to mitigate collateral tissue damage. The imaging device may be a C-arm X-ray device, a MRI device, an acoustic device or other device capable of producing CT-like images.

### TECHNICAL FIELD

The present application relates to a system and method of improving the medical treatment of patients using a combination of embolization and ablation therapies.

### BACKGROUND

Advances in percutaneous therapy have been made in the treatment of primary and metastatic tumors in the liver, spleen or kidneys. Studies have shown promising results using radiofrequency ablation (RFA) or chemoembolization (CE). Therapeutic response rates are reported to be as high as 79-100% for the treatment of renal cell carcinoma (RCC).

Some recent studies, however, suggest that there is a limited therapeutic response to RF ablation alone as tumor size increases or tumor geometry becomes complex (e.g., eccentric positioning). For example, RCCs show limited therapeutic response if substantial parts of the tumor extend into the renal sinus fat.

Another important factor relating to the success of RFA is that the volume of the thermal lesion created by RFA can substantially be influenced by blood flow. Thermal convection by the blood is an effective mechanism for heat transfer, and limits the amount of thermal energy that may be delivered to the lesion. Thus, blood flow limits the volume of coagulation necrosis occurring in the tumor. Devascularization of the tumor may also result in superior heat distribution throughout the lesion.

In the treatment of large hepatocellular carcinomas (HCC), the combined use of hepatic arterial occlusion is known to enhance the effect of RF ablation.

In presently used methods of treatment, in a first step, the patient is transferred to the angio-lab so as to perform angiography and CE. In a second step, the patient is then treated with RFA or other ablative technique in the CT department, because cross-sectional imaging will be required in order to perform ablation. In order to reduce procedure time for the patient (including preparation for angiography, embolization procedure, work up of this procedure, transport to the CT department, preparation for interventional CT, RF procedure, work up) during a single day and to let the patient recover from first procedure, the embolization and ablation procedures are performed on different days.

The existing workflow is time-consuming, with more than one department and more than one doctor team involved. Separate angiographic and CT imaging steps in the treatment procedure may require the administration of additional contrast material to the patient, which may impair renal function. During the procedures patients normally remain hospitalized, often for between 5-7 days, which adds additional costs.

### BRIEF SUMMARY

A system for treating a patient is disclosed, the system including an imaging device; a digital storage device; an embolization catheter; and, an ablation device. The imaging device is configurable to collect imaging data suitable to plan or monitor f embolization and ablation of a target tissue; and, the embolization catheter and the ablation device are usable where the patient is positioned on a same treatment table. The imaging device may be used to perform at least one of identify the tissue volume to be treated, evaluate the results of a treatment step, or guide the treatment devices.

In another aspect, a method of treating a patient includes obtaining image data of a patient; selecting a tissue volume of the patient for treatment; embolizing the tissue volume; and, ablating the tissue volume, wherein the steps of embolizing and ablating are performed while the patient remains on a treatment table. The step of embolizing may also include administering chemotherapy.

### BRIEF DESCRIPTION OF THE DRAWINGS

FIG. 1 is a block diagram of a system for treating a patient using a combination therapy;

FIG. 2 is flow chart of a workflow for treating the patient using the system of FIG. 1; and,

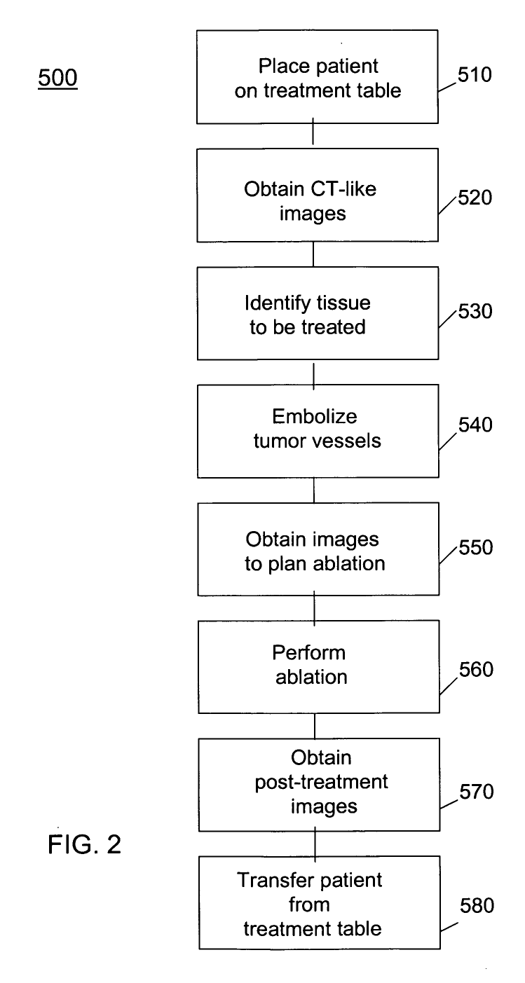

FIG. 3 is a flow chart of the workflow showing details of the embolization step of FIG. 2; and

FIG. 4 is a flow chart of the workflow showing details of the ablation step of FIG. 2.

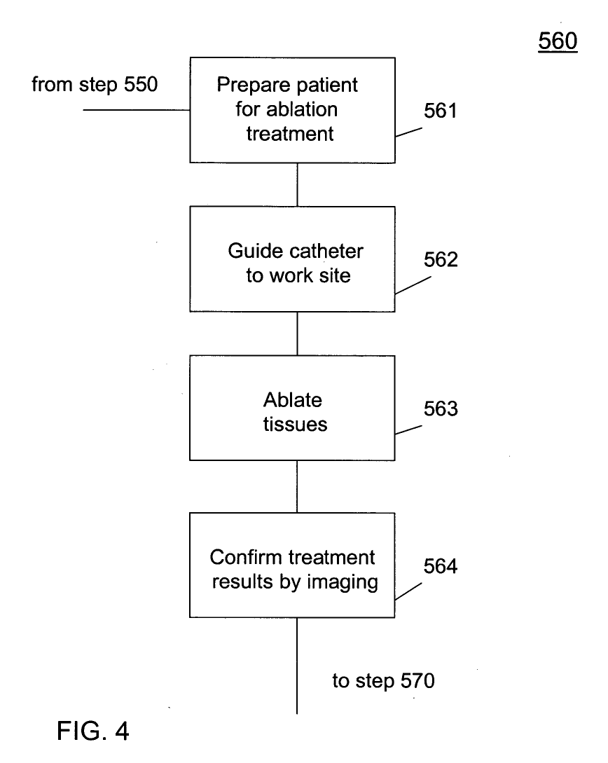

### DETAILED DESCRIPTION

Exemplary embodiments may be better understood with reference to the drawings. Like numbered elements in the same or different drawings perform equivalent functions.

In the interest of clarity, not all the routine features of the examples herein are described. It will of course be appreciated that in the development of any such actual implementation, numerous implementation-specific decisions must be made to achieve a developers' specific goals, such as consideration of system and business related constraints, and that these goals will vary from one implementation to another.

The examples of diseases, syndromes, conditions, and the like, and the types of examination and treatment protocols described herein are by way of example, and are not meant to suggest that the method and apparatus is limited to those named, or the equivalents thereof. As the medical arts are continually advancing, the use of the methods and apparatus described herein may be expected to encompass a broader scope in the diagnosis and treatment of patients.

An X-ray imaging modality may comprise an X-ray tube, high-voltage power supply, radiation aperture, X-ray detector, digital imaging system, system controller, as well as user control and display units. The X-ray detectors may be amorphous Selenium (a-Se), PbI2, CdTe or HgI2 detectors using direct detection and TFT technology, or indirect detectors as is known in the art, or may be subsequently be developed, to provide high resolution, high-dynamic-range real-time X-ray detection. The X-ray detector may be disposed diametrically opposed to the X-ray source and such that the plane of the detector is perpendicular to the axis of the X-ray source. This orientation may, for example, be maintained by attaching the X-ray source and X-ray detector to a C-arm, a U-arm or the like. The C-arm may be mounted to a robot so as to permit the X-ray source and detector to be oriented with respect to the patient.

The C-arm X-ray device is provided with an X-ray source and an X-ray detector, and may be operated to obtain fluoroscopic images, data suitable for the production of 2D, images, or computed tomography (CT)-like 3D images.

The use of the term CT-like data or CT-like images is understood to encompass data and images obtained by a CT device, a C-arm X-ray device or other tomographic imager as well, which may include magnetic resonance imaging (MRI).

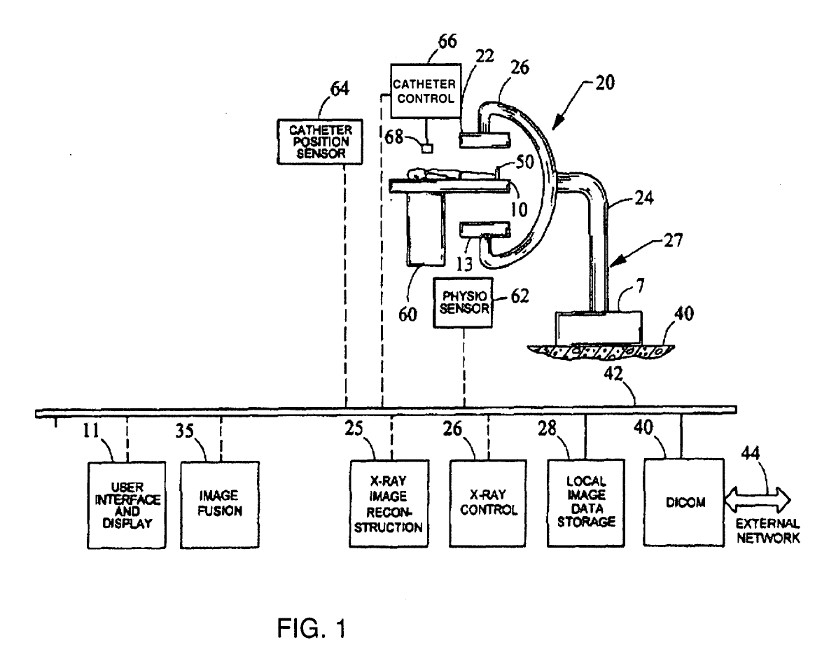

FIG. 1 shows a block diagram of an example of a system for the diagnosis and treatment of an illness by a use of a catheter. Depending on the procedure, a catheter may be used to, for example, deliver contrast material, embolitic or chemotherapy materials, or to perform ablation. Other embodiments of the system may include fewer than all of the devices, or functions, shown in FIG. 1. The data processing and system control is shown as an example, and many other physical and logical arrangements of components such as computers, signal processors, memories, displays and user interfaces are equally possible to perform the same or similar functions. The particular arrangement shown is convenient for explaining the functionality of the system.

The C-arm X-ray device 20 may comprise a C-arm support 26 to which an X-ray source 22, which may include a diaphragm to limit the field of view, and an X-ray detector 13 may be mounted so as to face each other about an axis of rotation. The C-arm 26 may be mounted to a robotic device 27 comprising a mounting device 7, and one or more arms 24 which are articulated so as to be capable of positioning the C-arm X-ray device with respect to a patient support apparatus 10. The robotic device 27 may be controlled by a control unit 11, which may send commands causing a motive device (not shown) to move the arms 24. The motive device may be a motor or a hydraulic mechanism. The mounting device may be mounted to a floor 40 as shown, to a ceiling or to a wall, and may be capable of moving in longitudinal and transverse directions with respect to the mounting surface.

The C-arm X-ray device 20 is rotatable such that a sequence of projection X-ray images may be obtained by an X-ray detector 13 positioned on an opposite side of the patient from the X-ray source 22, and the images may be reconstructed by any technique of processing for realizing computed tomographic (CT)-like, 3-D images, or 2-D cross-sectional images. Real-time fluoroscopic images may also be obtained from selectable orientations. A patient 50 may be positioned on a patient support apparatus 10. The patient support apparatus 10 may be a stretcher, gurney, or the like, and may be attached to a robot 60. The patient support apparatus 10 may also be attached to a fixed support or adapted to be removably attached to the robot. Aspects of the patient support apparatus 10 may be manipulable by the robot 60. Additional, different, or fewer components may be provided.

Where the robot 60 is integrated with the treatment and imaging apparatus, the position of the patient 50 may be adjusted, while maintaining registration of the body coordinates with respect to the treatment and imaging devices.

The devices and functions shown are representative, but not inclusive. The individual units, devices, or functions may communicate with each other over cables or in a wireless manner, and the use of dashed lines of different types for some of the connections in FIG. 1 is intended to suggest that alternative means of connectivity may be used.

The C-arm X-ray radiographic device 20 and the associated image processing 25 may produce angiographic and computed tomographic images comparable to, for example, CT equipment, while permitting more convenient access to the patient for ancillary equipment and treatment procedures and including the use of real-time fluoroscopic or CT-like images for diagnosis, catheter guidance, and the like. A separate processor 25 may be provided for this purpose, or the function may be combined with other processing functions. The various devices may communicate with a DICOM (Digital Communications in Medicine) system 40 and with external devices over a network interface 44, so as to store and retrieve image and other patient data.

Images reconstructed from the X-ray data may be stored in a non-volatile (persistent) storage device 28 for further use. The X-ray device 20 and the image processing attendant thereto may be controlled by a separate controller 26 or the function may be consolidated with the user interface and display 11.

The X-ray images may be obtained with or without various contrast agents that are appropriate to the imaging technology, tissue type, diagnosis and treatment protocol being used.

Additionally, a physiological sensor 62, which may be an electrocardiograph (ECG), a respiration sensor, or the like, may be used to monitor the patient 50 so as to enable selection of images that represent a particular portion of a cardiac or respiratory cycle as a means of minimizing motion artifacts in the images.

The treatment device 66 may be a catheter 68 which is introduced into the body of the patient 50 and guided to the treatment site by images obtained by the C-arm X-ray, or other sensor, such as a catheter position sensor 64. The catheter position sensor may use other than photon radiation; and, electromagnetic, magnetic and acoustical position sensors are known.

Apart from the sensors and positioning capabilities, the imaging, data processing, and controlling equipment may be located within the treatment room or remotely, and the remotely-located equipment may be connected to the treatment room by a telecommunications network. Aspects of the diagnosis and treatment may be performed without personnel, except for the patient, being present in any of the local treatment rooms.

The X-ray imaging device may be operated by rotating the C-arm such that the opposed X-ray source and X-ray detector traverse an angular range of about 180 degrees about an axis perpendicular to the plane of the C-arm. A 3D or CT-like image may be reconstructed from the detected X-ray data, or 2D images or fluoroscopic images may be reconstructed in various image planes. The algorithmic and measurement aspects of computed tomography images are being improved, and the processing of the images obtained by the imaging devices are expected to continue to improve in resolution and dynamic range, speed, and in reduction of the X-ray dosage. Such improvements also may include segmentation, using tissue-specific absorption characteristics to automatically differentiate between tissue types, automated registration of the 2D or fluoroscopic images with 3D or CT-like images in arbitrary orientation planes, and the like.

The term “X-ray” is used to describe any device that uses ionizing radiation to obtain data characterizing the opacity of a path through a patient, or part thereof, regardless of the wavelength or source of the radiation used. Equivalently, devices using non-ionizing radiation, such as magnetic resonance imaging (MRI) and acoustic tomography may also be used to image the area to be diagnosed and treated, and to guide the catheter. Collectively these may be called “imaging modalities” although the term X-ray is often used in this specification to represent the general class of imaging devices for obtaining information about the interior of a patient. Where the term “catheter” is used, it is intended to represent any treatment apparatus introduced into the patient's body, and may also include contrast agents introduced intra-operatively to visualize a vascular structure, a tumor, or the like. As such, a catheter may be used to administer a contrast agent, a stain, embolitic or chemotherapy material, or ablation energy. The catheters used may deliver a class of treatment such as embolitic and chemotherapy, or be different for each treatment type.

Flat detector fluoroscopic imaging systems, such as the Siemens C-arm “Axiom” (available from Siemens AG, Munich, Germany) are capable of acquiring CT-like data sets of the subjects, and it is becoming increasingly common to acquire 3D or CT-like datasets with contrast injected into specific vessels to enhance diagnosis or enable planning for interventions.

The positioning of a catheter inside a patient, and the manipulation of the catheter position to administer treatment is facilitated by the use of real-time fluoroscopic images of the patient. Alternatively, the position of the catheter or other apparatus may be measured by acoustic or magnetic means, and superimposed on a fluoroscopic or 2-D X-ray image, and the image may be a previously captured image, or based on previously acquired data. The catheter position may also be shown on CT-like images in multiple views.

A catheter locating system (for example, U.S. Pat. No. 5,042,486, “Catheter Locatable with Non-Ionizing Field and Method for Locating Same”) can be integrated into the system. The catheter may be provided with position sensors, such as electromagnetic sensors or magnetic sensors. Thus the tip of the catheter, in particular, can be detected without emitting continuous X-rays and the motion thereof can be followed and displayed with respect to a previously obtained image by adding the catheter position to the images synthetically.

Catheter-based ablation techniques generally fall into two categories: (1) cold-based procedures where tissue cooling is used (cryoablation) and (2) heat-based procedures (thermoablation) where a high temperature is used.

In another aspect, a catheter may be used to deliver chemotherapy to a local volume, and this may be combined with embolization of the arteries feeding the tumor. When catheter-delivered chemotherapy is used, drug concentrations in the tumor may be one or two orders of magnitude greater than are achieved by infusion alone, and the drug levels may persist for a substantially longer time. Embolization renders the tumor ischemic, depriving it of nutrients and oxygen, leading to necrosis. Embolization may be performed by devices, such as coils, by particles, a gelatin sponge, or by chemicals. Such chemicals as polyvinyl alcohol, ethanol mixed with iodized oil, or other embolizing agents may be used. The chemotherapy may be selected from the chemotherapy agents known to be effective with respect to the tumor type being treated, although off-label used of drugs of all kinds are known and are not excluded; the use of a particular treatment being a prerogative of the physician.

In another aspect, a catheter or laparoscopic technique may be used to place a balloon in a position such that, for example, a cooling solution may disposed in a region between the tumor being treated and another structure that may be damaged by the heat. This sometimes used, for example, the treatment of prostate cancer, where damage to the rectum may occur. An example of such a technique is hydro-dissection, in which a window of sterile water or similar non-ionic solution (e.g., dextrose in water) is injected through a fine needle inserted between the tumor and the organ or tissue deemed at risk. The injected fluid serves to separate the protected tissues from the tissues being treated, so as to mitigate pr prevent collateral damage.

Cryoablation uses a hollow needle (cryoprobes) through which cooled, thermally conductive, gases or fluids are circulated. The cryoprobe may inserted into, or placed adjacent to, tissue which is to be ablated. When the probe is in place, a cryogenic freezing unit removes heat (“cools”) from the tip of the probe and, as a consequence, from the surrounding tissues. The temperature may be reduced to as low as about −75° C. in a local area and an iceball may be formed.

Ablation is believed to occur in the frozen tissue by at least three mechanisms: formation of ice crystals within cells thereby disrupting membranes, and interrupting cellular metabolism processes; coagulation of blood thereby interrupting blood flow to the tissue and resulting in ischemia and cell death; and, induction of apoptosis. The most common application of cryoablation is to ablate solid tumors found in the lung, liver, breast, kidney and prostate gland. Although sometimes applied through laparoscopic or open surgical approaches, most often cryoablation is performed percutaneously (through the skin and into the target tissue containing the tumor).

Thermoablation may include RF ablation (RFA), high focused ultrasound (HIFU), laser ablation and the like, which use technologies that may be capable of delivering energy to targeted tissues, with minimal effect on surrounding tissues.

In RFA therapy, electromagnetic energy is delivered through a metal tube (probe) inserted into tumors or other tissues. When the probe is in place, metal prongs may open out to extend the reach of the therapy. RF (radio frequency) energy causes atoms in the cells to vibrate and create friction. This generates heat (up to about 100° C.) and leads to the death of the cells.

In HIFU therapy, ultrasound beams are focused on the target diseased tissue, and due to the significant energy deposition at the beam focus, temperature within the tissue rises to from about 65° to about 85° C., and is believed to destroy the diseased tissue by melting the lipids in the cell membrane, denaturing proteins, and by coagulation. The focal volume of the ultrasound beam is usually small, perhaps the size of a grain of rice. Consequently, a larger volume may be treated by repeatedly applying the HIFU beam to adjacent areas, or areas individually selected by an imaging technique.

Each of the ablation techniques has differing requirements for the precision and accuracy of delivery of the energy or cold to the tissues to be treated. However, the efficacy of the treatment depends on delivering the catheter treatment to the tissue while minimizing the collateral damage to normal adjacent tissues and structures.

Additional contrast material may be administered to aid in visualization of the process in needed, and where robotic control of the catheter has been used, or the catheter location has been tracked by acoustic or magnetic, or fluoroscopic techniques, the volumetric dosage of the ablation technique may be determined so as to appropriately deliver the treatment with minimal damage to surrounding tissues.

Performing the steps of obtaining 3-dimensional images of the patient, which may include angiographic images, and performing embolization or chemo-embolization and ablation in a substantially uninterrupted sequence may reduce the stress on the patient as well as save time and reduce cost, with respect to existing workflow methodologies. A C-arm X-ray device, such as the AXIOM Artis DynaCT (Siemens AG, Munich, Germany) is capable of obtaining CT-like image data and conventional radiographic and fluoroscopic data, while permitting convenient access to the patient for the use of other equipment such as the catheter treatments administered as previously described.

A workflow method for the treatment of, for example, hepatic, prostrate or renal cancers may include the steps of: performing an angiographic study to identify and localize the diseased tissues to be treated; blocking blood flow to the identified tissues using a catheter based procedure such as embolization, chemoembolization, embolization with devices, or temporary blockage with an occlusion balloon; and, after the embolization has been performed, a catheter, laproscopic based or minimally invasive technique of performing ablation may be used to further the tissue necrosis process. The patient may remain on the treatment table throughout the treatment process.

Where the treatment table is a robotically operated device, the position of the patient may be adjusted between or during the various steps of the treatment, while maintaining registration of the coordinates of the various treatment devices with respect to the patient anatomy. CT-images, or fluoroscopic images, which may be roadmap images, may be used to accurately position the catheter with respect to the tissue to be treated.

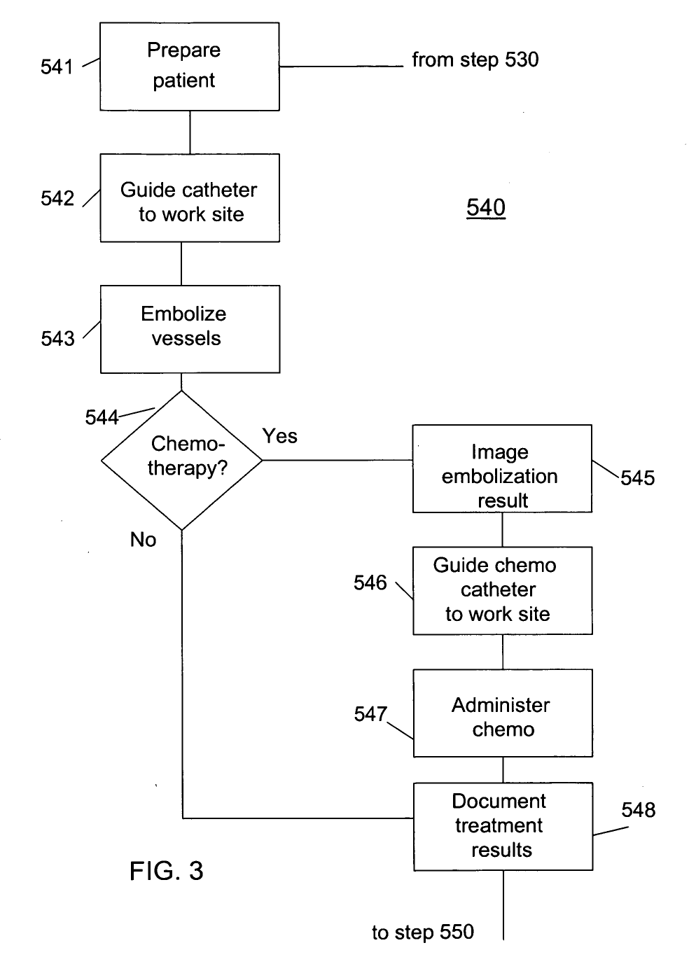

In an example of a clinical workflow for performing the method, FIG. 3 illustrates a patient treatment protocol (step 500) that includes embolization and ablation. The patient is placed on a treatment table (step 510), and this step may be performed by a robot or manually. The treatment table may already be in a suitable position for use by a system such as shown in FIG. 1, or be moved to such a position. The imaging modality is employed to obtain CT-like images (step 520), which may be with or without administration of contrast agents. The images obtained in this step may be CT-like, be 3 dimensional or be 2 dimensional. The images may be used both to diagnose the syndrome, and to plan the subsequent treatment. Previous images may have been obtained in earlier diagnostic steps, and such images may have been stored in a data base. Pre-operative images may be retrieved using protocols such as DICOM, and may be fused or compared with the present images.

A variety of image processing and display techniques may be used, depending on the availability of data, the processing algorithms available in the specific equipment, and the preferences of the treatment team based on current best practice. These may include 3D/2D image fusion, segmentation, fluoroscopic road mapping, and the like. Where the use of images are described in this workflow, images from any suitable imaging modality may be used, including CT, C-arm X-ray, MRI and acoustic.

The tissue to be treated may be identified by the medical team (step 530), and the specific sites for performing embolization selected. The tissues are embolized using a catheter administered agent (step 540), and the step of embolization may include the administering of chemotherapy by dispensing the chemotherapy agent directly into the tissues to be treated, typically through the vascular system. The administration of the chemotherapy may be contemporaneous with the embolization. Where embolization is effected by a balloon, the blockage of the vessel is temporary, and may be maintained during the administration of chemotherapy, and during a subsequent ablation step. In such a circumstance, the embolization by inflatable balloon may be discontinued after the ablation has been performed. As ablation also results in coagulation of the blood vessels, the overall embolization effect may be maintained for the area that has been ablated.

The patient may remain on the treatment table after the embolization of the tumor, and additional images may be obtained by the imaging modality (step 550) to conform the effectiveness of the embolization. The patient may be repositioned by the robotic system, or manually. Depending on the type of repositioning, additional imaging may be needed to provide the appropriate visualization and coordinate registration for guidance of the ablation catheter.

Ablation of the selected tissues may be performed using one of the techniques described so as to cause tissue necrosis (step 560). This process may be monitored fluoroscopically or by 3-D rotational imaging (e.g., CT-like) so as to confirm the location of the ablation catheter, and the extent of the region being affected. The positioning of the catheter may be performed using the images obtained by the imaging modality, by magnetic or acoustic sensors, or a combination of the techniques. The manipulation of the catheter may be performed in whole or in part by a robotic device. The position of the catheter may be shown on previously obtained images using image processing. In an aspect, the combination of an imaging modality with other catheter positioning techniques may reduce the total radiation dose administered to the patient during the treatment.

When desired, post-treatment images may be obtained (step 570) to confirm the results of the treatment and serve as a baseline for follow-up studies. However, this step may be omitted where the images obtained during the treatment are judged to be sufficient for this purpose.

At the completion of the treatment, the patient is prepared for transfer from the treatment table (step 580) and may be moved either by a robotic device or manually to a bed or other recovery position.

In an aspect, the embolization step 540 may include preparing the patient for catheter insertion (step 541), where the preparation may include gaining access to the vascular system through a small incision, or the like, inserting the catheter, and moving the catheter to the vicinity of the work site. When the catheter is in the vicinity of the work site, the positioning and guidance of the catheter may be by any method, which may include one or more of the imaging modality, magnetic, or acoustic sensors, or the like (step 542). When the catheter is suitably placed, the embolization chemicals or inflated balloon may be used to block blood flow to the tissue to be treated (step 543). At this time, the treatment workflow takes different paths (step 544), depending on whether chemotherapy is being administered. Where only embolization is being performed prior to ablation, the workflow of step 540 would conclude by removal of the catheter, and possibly obtaining images from the imaging modality that would confirm the results of the embolization treatment. In the case of balloon embolization, which is a temporary procedure, the catheter may remain in place until the conclusion of the ablation step. The catheter may also be left in place so as to be able to deliver contrast agents for purposes of evaluating the results of ablation.

When chemotherapy is part of the workflow, an image may be evaluated to confirm the results of the previously performed embolization (step 545). Another catheter may be guided into the work area (step 546) so as to administer chemotherapy agents (step 547), or the same catheter as used for embolization may be employed. As in the case of embolization alone, post-procedure images may be obtained for evaluation (step 548).

In another aspect, the ablation step of the work flow may include preparing the patient for use of the ablation catheter (step 561). The ablation catheter may be inserted, for example, percutaneously, and may be guided to the work site by methods similar to those used for the embolization catheter (step 562). As the ablation catheter may have a form such as a needle or probe, and may be substantially rigid, the guidance of the ablation device may be by a robot. The ablation device may also be a HIFU and the guidance may also be by a robotic device, or by beam steering of the acoustic transducer as the individual volumes treated by each actuation of the device are likely to be smaller than the overall volume to be treated.

Additional devices such as needles may be used to administer fluids for hyrodissection, or other techniques may used to mitigate collateral damage due to the ablation energy.

The tissues are ablated (step 563) by the introduction of energy (thermoablation) or the removal of energy (cryoablation). The progress of the ablation may be monitored by the used of the imaging modality, with or without contrast, as the ablation results in prompt necrosis of the tissues, and corresponding changes in the images. The extent of the ice ball formed by cryoablation may be determined by acoustic or X-ray techniques.

At the conclusion of the ablation process, the results may be documented by imaging (step 564).

For the purposes of this specification, the terms pre-operatively or equivalent may be considered to represent a time where diagnosis is being performed, including obtaining such data as imaging data, with or without contrast, biopsies, or the like, at any time preceding the treatment. During this period, the procedures may be non-invasive or minimally invasive, as is known in the art, such as the insertion of a measurement catheter or the administration of contrast agents, or the like. Intra-operatively may be considered to represent the time where a specific course of treatment is being administered, based on the pre-operative data. The course of treatment may be modified during the intra-operative procedure based on the results being obtained and other considerations. Although data for CT-like images is often obtained during the pre-operative period, this is due primarily to the time needed to collect and process the data using existing commercial equipment. The distinction between the pre-operative and intra-operative periods is likely to be reduced or eliminated as processing speeds increase. As such, the terms pre-operative and intra-operative should not be considered to be disjoint time frames.

While the methods disclosed herein have been described and shown with reference to particular steps performed in a particular order, it will be understood that these steps may be combined, sub-divided, or reordered to from an equivalent method without departing from the teachings of the present invention. Accordingly, unless specifically indicated herein, the order and grouping of steps is not a limitation of the present invention.

Although only a few examples of this invention have been described in detail above, those skilled in the art will readily appreciate that many modifications are possible without materially departing from the novel teachings and advantages of the invention. Accordingly, all such modifications are intended to be included within the scope of this invention as defined in the following claims.

What is claimed is:

1. A system for treating a patient, comprising:
an imaging modality;
an embolization catheter; and
an ablation device,
wherein the imaging modality is configurable to collect imaging data suitable to plan or monitor effects of embolization and ablation on a tissue volume; and, the embolization catheter and the ablation device are usable where the patient is positioned on a same treatment table.

2. The system of claim 1, wherein the imaging modality is a C-arm X-ray device.

3. The system of claim 1, wherein the embolization catheter is also a chemotherapy catheter.

4. The system of claim 1, wherein the embolization catheter is a balloon catheter.

5. The system of claim 1, wherein the ablation device is a thermoablation device.

6. The system of claim 1, wherein the ablation device is High Focused Ultrasound (HIFU) device.

7. The system of claim 1, wherein the ablation device is a cryoablation device.

8. The system of claim 1, further including a magnetic sensor for determining a position of at least one of at tip of the embolization catheter or a tip of a probe of the ablation device.

9. The system of claim 1, wherein the imaging modality is configured to produce CT-like images.

10. The system of claim 1, wherein the imaging modality is configured to produce 2-D or fluoroscopic images.

11. The system of claim 1, wherein the imaging modality uses acoustic energy.

12. A method of treating a patient, the method comprising:
obtaining image data of a patient;
selecting a tissue volume of the patient for treatment;
embolizing the tissue volume; and
ablating the tissue volume,
wherein the steps of embolizing and ablating are performed while the patient remains on a treatment table.

13. The method of claim 12, wherein the step of embolizing includes:
guiding a catheter to a work area;
administering an embolizing chemical or device; and,
subsequently administering chemotherapy.

14. The method of claim 12, where the step of embolizing includes a step of administering chemotherapy.

15. The method of claim 12, wherein the step of ablating is performed subsequent to the step of embolizing.

16. The method of claim 12, wherein the step of embolizing includes inserting an inflatable balloon so as to temporarily obstruct blood flow to the tissue volume.

17. The method of claim 12, wherein the step of ablating is performed by guiding the tip of a treatment apparatus to one of deliver or remove heat energy from at least a portion of the tissue volume to be ablated.

18. The method of claim 12, wherein the step of ablating is performed by focusing acoustic energy in at least a portion of the tissue volume to be ablated.

19. The method of claim 12, wherein the position of a catheter for performing at least one of embolization or ablation is determined by acoustic or magnetic devices.

20. The method claim 12, wherein the patient or a device for performing embolization are manipulable by a robot.

21. The method of claim 12, wherein the patient or a device for performing ablation are manipulable by a robot.
